(19) United States
(12) Patent Application Publication
(10) Pub. No.: US 2011/0059177 A1
(43) Pub. Date: Mar. 10, 2011

(54) Cardioplegia Solution for Cardiac Surgery

(76) Inventor: Hemant Thatte, Medfield, MA (US)

(21) Appl. No.: 12/867,408

(22) PCT Filed: Feb. 10, 2009
(86) PCT No.: PCT/US09/00834

§ 371 Date: Nov. 1, 2010

Related U.S. Application Data
(60) Provisional application No. 61/065,952, filed on Feb. 15, 2008.

(51) Int. Cl. A61K 33/14; A61K 31/4418; A61K 31/255; A61K 31/16; A61K 31/65; A61K 31/7004; A61K 9/14; A61P 9/00; A61P 41/00

(52) U.S. Cl. 424/489; 514/356; 514/517; 514/627; 514/152; 424/665; 514/23

(57) ABSTRACT

The invention relates to improved cardioplegia solutions. The invention provides cardioplegia solutions and compositions that produce a readily reversible, rapid electrochemical arrest with minimal tissue ischaemia. The cardioplegia solutions and compositions are used for arresting, protecting and/or preserving organs, in particular the heart during open-heart surgery, transplanting, cardiovascular diagnosis or therapeutic intervention.

### RELATED APPLICATIONS

This application claims the benefit of U.S. Ser. No. 61/065,952, filed Feb. 15, 2008, which is incorporated herein by reference in its entirety.

### FIELD OF THE INVENTION

The invention relates to compositions for arresting, protecting and/or preserving organs, in particular the heart, during open-heart surgery, cardiovascular diagnosis or therapeutic intervention.

### BACKGROUND OF THE INVENTION

Current cardioplegia solutions may be linked to myocardial stunning, ventricular arrhythmias, ischaemic injury, endothelial cell swelling, microvascular damage, and cell death. Accordingly, there is a need to develop cardioplegia solutions that produce a readily reversible, rapid electrochemical arrest with minimal tissue ischaemia.

### SUMMARY OF THE INVENTION

The invention relates to improved cardioplegia solutions. The invention provides cardioplegia solutions and compositions that produce a readily reversible, rapid electrochemical arrest with minimal tissue ischaemia. The cardioplegia solutions and compositions are used for arresting, protecting and/or preserving organs, in particular the heart during open-heart surgery, transplanting, cardiovascular diagnosis or therapeutic intervention. The cardioplegia solutions and compositions comprise a physiological salt solution and optionally one or more of the following compositions: a substrate for the production of ATP, a calcium channel blocker, a vasorelaxant, a reagent that buffers intracellular acidity, an antioxidant, and/or an antibiotic.

The invention provides a cardioplegia solution comprising a physiological salt solution and beta-alanine. Optionally, the cardioplegia solution comprises a physiological salt solution, beta-alanine, and one or more ingredients selected from the group consisting of anandamide, minocycline, lacidipine, potassium chloride, magnesium chloride, and D-glucose.

The invention also provides a cardioplegia solution comprising a physiological salt solution, beta-alanine, anandamide, lacidipine, taurine, and minocycline. The invention also provides a cardioplegia solution comprising a physiological salt solution, beta-alanine and anandamide. In another aspect, the invention provides a cardioplegia solution comprising a physiological salt solution, beta-alanine and minocycline. In yet another aspect, the invention provides a cardioplegia solution comprising a physiological salt solution, beta-alanine and lacidipine.

Anandamide, minocycline, and/or lacidipine may be present or absent in the solution based on the discretion of the surgeon.

The invention provides a cardioplegia solution comprising a physiological salt solution further comprising at least one, at least two, at least three, at least four, or at least five of a composition selected from the group consisting of a calcium channel blocker, a vasorelaxant, a reagent that buffers intracellular acidity, an antioxidant, and an antibiotic.

In one aspect, the invention provides a cardioplegia solution comprising a physiological salt solution and a calcium channel blocker. Optionally, the calcium channel blocker is lacidipine.

The invention also provides a cardioplegia solution comprising a physiological salt solution and a vasorelaxant. In one aspect, the vasorelaxant is anandamide.

The invention also provides cardioplegia solution comprising a physiological salt solution and an agent that buffers intracellular acidity. Preferably, the reagent that buffers intracellular acidity is beta-alanine.

In yet another aspect, the invention provides a cardioplegia solution comprising a physiological salt solution and an antioxidant. Optionally, the antioxidant is taurine.

The invention also provides a cardioplegia solution comprising a physiological salt solution and an antibiotic. Preferably, the antibiotic is minocycline.

The cardioplegia solutions of the invention optionally comprise potassium chloride, magnesium chloride, and/or D-glucose.

In one aspect, the cardioplegia solution of the invention comprises:

0.01-3.00 L distilled water

0.1-0.5 gm/L calcium chloride

0.1-0.5 gm/L potassium chloride

0.01-0.25 gm/L potassium phosphate (monobasic)

0.05-0.5 gm/L magnesium chloride (hexahydrate)

0.05-0.5 gm/L magnesium sulfate (heptahydrate)

5-10 gm/L sodium chloride

0.25-0.75 gm/L sodium bicarbonate

0.01-0.1 gm/L sodium phosphate (dibasic; heptahydrate)

0.5-2.5 gm/L D-Glucose

0.25-2.5 gm/L adenosine

1-5 gm/L glutathione (reduced)

0.01-0.5 gm/L ascorbic acid

0.5-2.5 gm/L L-Arginine

0.1-1 gm/L L-Taurine

0.1-1 gm/L Beta-alanine

L-Histidine

1-5 gm/L L-Carnosine

0.1-0.5 gm/L creatine monohydrate

0.0001-0.005 gm/L anandamide

0.1-1 gm/L minocycline

0.00001-0.001 gm/L lacidipine

Tris-hydroxymethyl aminomethane (THAM) is used to adjust pH. Preferably, the cardioplegia solution of the invention does not contain insulin. Also within the invention are kits comprising the cardioplegia solutions described herein.

In one aspect, the cardioplegia solution of the invention comprises water clusters in a nanometer range of size. Optionally, the solution of the invention is nano-sized to increase the efficiency of traversing the cellular membrane. Nano-sizing refers to the reduction of the particle size to the sub-micron range, with the final particle size typically being 1-10 ηm. The reduction of particle size leads to a significant increase in the efficiency of the solution in traversing the cellular membrane. In one aspect, the efficiency is increased such that at least 20%, at least 25%, at least 50%, at least 75%, or at least 100% of the solution traverses the cellular membrane.

The invention provides for nano-sizing the solution of the invention prior to use in the methods described herein. Alternatively, the invention provides for nano-sizing the water prior to adding the other compounds/reagents of the solution. In yet another aspect, the invention provides for nano-sizing the water and nano-sizing each compound/reagent of the solution separately prior to mixing in solution.

In one aspect, the composition comprises water packets or water clusters in a nanometer range of size. Optionally, the water packets or water clusters are 1-10 ηm, 1-25 ηm 25-50 ηm, 50-75 ηm, 75-100 ηm, 100-200 ηm, 200-500 ηm, or 500-999 ηm.

In one aspect, the solutions provided by the invention are administered antegrade. Alternatively, the solutions are administered retrograde.

In another aspect, the solutions presented are mixed with blood prior to use in order to form blood cardioplegia solutions. Optionally, the blood:solution ratio is 5:1, 4:1, 3:1, 2:1 or 1:1. Alternatively, the blood:solution ratio is 1:2, 1:3, 1:4, or 1:5.

In one example, the ingredients of the invention are in powder form and reconstituted prior to use. Preferably, the ingredients are reconstituted in water.

Cited publications are incorporated herein by reference. Both the foregoing general description and the following detailed description and examples are exemplary and explanatory only and are not restrictive of the invention as claimed.

### DETAILED DESCRIPTION OF THE INVENTION

There are over 800,000 open-heart surgery operations each year in the United States and approximately 1,000,000 in Europe. During cardiovascular surgery and cardiac valve surgery (commonly known as open heart surgery), it is often necessary to arrest the pumping activity of the heart by inducing heart paralysis or “cardioplegia.” The cardioplegia solution discontinues the beating of the heart in a manner that minimizes damage to the heart's myocardium and renders the heart motionless so that a surgeon may operate. The heart may be arrested for up to 3 hours during open-heart surgery. High potassium cardioplegia (in excess of 15-20 mM) has been the basis of myocardial arrest and protection for over 40 years. Currently, the majority of solutions used contain high potassium including the widely used St Thomas No. 2 Hospital Solution which generally contains 110 mM NaCl, 16 mM KCl, 16 mM MgCl2, 1.2 mM CaCl2 and 10 mM NaHCO3 and has a pH of about 7.8. Notwithstanding hyperkalemic solutions providing acceptable clinical outcomes, recent evidence suggests that progressive potassium induced depolarisation leads to ionic and metabolic imbalances that may be linked to myocardial stunning, ventricular arrhythmias, ischaemic injury, endothelial cell swelling, microvascular damage, cell death and loss of pump function during the reperfusion period. In some cases, high potassium induced ischaemia has been reported to have damaged smooth muscle and endothelial function.

In almost every major cardiac surgical operation performed today, the blood supply to the heart is interrupted, either regionally or globally, thus setting the stage for the onset of time-dependent myocardial ischemia the severity of which is a major determinant of the patient's postoperative outcome. To avoid severe and irreversible myocardial ischemic damage during cardiac surgery, a compendium of intra-operative myocardial protection techniques are routinely employed, which include the administration of a cardioplegic solution to the heart throughout prolonged periods of global interruption of blood flow. Despite voluminous experimentation in the field of myocardial protection, inadequate protection from peri-operative myocardial injury continues to be one of the major causes of early and late mortality following cardiac surgery. Online measurement of myocardial tissue pH provides a reliable measure of myocardial tissue ischemia in real time, and has been employed in selected patients undergoing cardiac surgery. Experience with nearly 700 patients in whom myocardial pH was continuously measured during cardiac surgery has revealed a wide variation among patients in the magnitude of myocardial tissue acidosis observed during the course of a cardiac surgical operation (Tantillo M B and Khuri S F, 1993, In Piper H M, Preusse C J (eds): Ischemia-reperfusion in Cardiac Surgery, Netherlands, Kluwer Academic Publishers; Khuri S F et al. 1983, J Thorac Cardiovasc Surg, 86:667-78).

Studies have implicated acidosis as a central component of apoptosis in a number of systems (Webster K A et al., J Clin Invest, 104:239-252). Acidosis may be the actual signal that initiates apoptosis in cardiac myocytes subjected to hypoxia. Cardiac myocyte cell death by apoptosis accompanies heart diseases of both ischemic and non-ischemic origin. However, little is known about the initiating events and the mechanisms of apoptosis in ischemic heart disease and congestive heart failure. Circulating neurohumoral factors, such as atrial natriuretic peptide, TNF-α, angiotensin II, norepinephrin and endothelin, are elevated in chronic congestive heart failure and may contribute to apoptosis. Mechanical factors, such as wall stress and stretch may also play a role, and there is strong evidence for a direct involvement of oxygen free radicals generated during ischemia reperfusion. Other studies have shown that intracellular acidification is frequently associated with apoptosis in some cell types although the relationship between the two is not clear. All ischemic conditions involve reduced washout of waste metabolites, consequently acidosis is a common feature of ischemic tissues. In ischemic myocardial tissue, the buildup of extracellular acid influences other ion channel activities and can have profound effects on [Ca²⁺]_{I}, [pH]_{I}, and contractility.

### General Purpose of the Cardioplegia Solutions

Cardiopulmonary bypass (CPB) or extracorporeal circulation is a highly sophisticated life support system instituted by artificial means in cardiovascular surgery. The system performs the functions of the heart (circulation of blood) and lungs (gas exchange) during cardiovascular surgical procedures. As blood is anticoagulated and drained via the venous cannula in the right atrium then pumped through the extracorporeal circuit, it is oxygenated, filtered, cooled or warmed, and returned by means of an arterial cannula to systemic circulation. Thereby, vital organs and body tissues are perfused, and thus they remain viable despite temporary interruption of heart and lung function. In most CPB procedures, ascending aorta is cross-clamped, thereby excluding the coronary arteries from the extracorporeal circuit. In order to stop the beating of the heart, a high potassium cardioplegic solution is used to temporarily paralyze the heart in the diastolic state during the course of the surgery. The solution is mixed with blood and infused into the coronary circulation to induce and maintain an arrest during the surgery. This is one of the options for managing the heart during On Pump CPB. There are various types of cardioplegia solutions available that contain excess of extracellular potassium ions that function by abolishing the transmembrane gradient of potassium across the myocyte cell membranes, thereby inhibiting repolarization. As a result, the heart remains flaccid in diastolic arrest with no electrical or mechanical activity. This inactivity is reversed over time (˜15 to 20 minutes) by normal metabolic mechanisms present within the cell; unless otherwise maintained in arrested state for prolonged period using the cardioplegia solution, until completion of the surgery. However, as the heart remains in an unnaturally arrested state, what happens during this time to the biochemical and physiological processes, and tissue viability, ultimately defines the recovery of the heart, and long-term patient outcomes. However, clinical and research evidence shows that none of the currently available cardioplegia solutions are able to protect the heart from ischemia-reperfusion and acidosis, mediated apoptotic and/or necrotic injury. The outstanding ability of the solution of the invention to protect the biochemical and physiological function and the tissue viability of explanted organs over extended periods, leads to the hypothesis that the solution of the invention could operate as a cardioprotective, cardioplegia and CPB pump priming solution. Results have shown the hypothesis to be correct, in that, the cardioplegia solution of the invention is able to protect heart tissue, cardiac endothelium of the coronaries, and cardiac myocytes from ischemia-reperfusion and acidosis induced apoptotic injury.

### Protection of Cardiac Myocytes and the Endothelium

The solution of the invention is a physiological salt solution comprising ascorbic acid and glutathione as reducing agents and free radical scavengers and L-arginine as a substrate for eNOS activity. Ascorbic acid, an antioxidant is known to scavenge reactive oxygen species and thus demonstrate sparing action on cellular glutathione and alpha tocopherol in the plasma membranes. It also preserves endothelium derived nitric oxide bioactivity by scavenging superoxide anions, and increases eNOS activity, perhaps due to prevention of oxidation of tetrahydrobiopterin, an eNOS cofactor. Ascorbic acid reduces platelet activation and leukocyte adhesion, and decrease endothelial layer permeability via ascorbic acid-mediated collagen synthesis. Similarly, ascorbic acid reduces smooth muscle cell proliferation, increases prostacyclin production and reduces lipid peroxidation. Similarly, the role of glutathione as a cellular reducing agent and antioxidant, scavenging reactive oxygen species has been well established. Glutathione has also been shown to increase L-arginine transport in endothelial cells and increases eNOS activity, NO generation and coronary vasodialation. Glutathione also increases the formation of biologically active S-nitrosoglutathione that can contribute NO and is also known to potentiate vasodilatory effects of nitroglycerin. The role of L-arginine in antioxidative therapy is not well established, but its role as the substrate for NO synthase has been well known. Oral administration of L-arginine has also been shown to decrease neutrophil-endothelial cell interactions in inflamed vessels. Therefore, multifaceted effects of ascorbic acid, glutathione and perhaps L-arginine are expected to protect the cardiac myocyte and coronary vascular structure and functions and attenuate ischemia-reperfusion and acidosis-induced apoptosis. In one aspect, the cardioplegia solution of the invention is mixed with blood in a 4:1 ratio (4 parts blood:1 part solution) to prevent and/or reverse acidosis/ischemia and reperfusion induced detrimental effects in cardiac myocytes and coronary vasculature.

During the heart surgery, myocardial perfusion can be interrupted for brief periods (15 to 20 minutes) with relative safety. This is due to the unique ability of the heart muscle cells to utilize anaerobic pathways for energy production. Because adenosine triphosphate (ATP) stores are insufficient for maintaining cardiac contraction, high-energy phosphates produced by anaerobic metabolism can sustain cell viability. If perfusion with cardioplegic solution, which is mildly hypothermic (25° C.), to temporarily paralyze the heart is not performed, contractions or ventricular fibrillation would continue for a variable period during the surgical operation and thus deplete energy stores. Therefore, administration of cardioplegic solution after aortic cross-clamping preserves ATP stores and reduces the development of intramyocardial acidosis and production of carbon dioxide by stopping the heart. Multidose cardioplegia helps maintain minimal ATP stores required for tissue safe keeping by permitting more effective anaerobic metabolism, perhaps by continuous replenishing of energy source and metabolite washout. Because the arrested heart remains flaccid due to cardioplegia, and thus there is no resultant flow through the coronary arteries or coronary sinus, the operative procedures become ideal because of a stable surgical field in addition to the decreased metabolism due to hypothermia. Also, because of continuous aortic root venting, no coronary blood flow should occur after administration of cardioplegic solution because it would wash out the solution and allow electrical or even mechanical activity to resume. There are various cardioplegia solutions available in the market today, such as Melrose solution, amino acid-enriched solution that contains mono-sodium L-glutamate (MSG) and monosodium L-aspartate (MSA), surgical cardioplegic solution, containing Dextrose 5%, Dextrose 50%, Tham and citrate phosphate dextrose (CPD), Plegisol solution, St. Thomas' solution, Bretsschneider solution, Buckberg solution and blood cardioplegia. However, none of these solutions are able to protect the cardiac myocyte and coronary endothelium from ischemia reperfusion injuries, and totally preserve the functionality of anastomised grafts.

### Reperfusion

After completion of the surgical procedure, the early moments of reperfusion of the coronary circulation after removal of aortic cross-clamping is a critical period, especially after revascularization of acutely ischemic myocardium. To prevent myocardial damage, it is important to carefully normalize the physical, biochemical and physiological processes during reoxygenation, and excessive pressure buildup during early reperfusion.

A number of advantages are associated with the cardioplegia solution of the invention. The solution delivers free radical scavengers, reducing agents, and minocycline to cardiac myocytes and the coronary vasculature. These compounds counteract hyper-oxygenation, free radical scourge (damage), and the induction of apoptosis, e.g., apoptosis due to calcium overload and mitochondrial pore transition after release of aortic cross clamping and reperfusion of the ischemic tissue.

### Cardioplegia Solutions

The invention provides improved cardioplegia solutions and compositions that produce a readily reversible, rapid electrochemical arrest with minimal tissue ischaemia. The cardioplegia solutions and compositions are used for arresting, protecting and/or preserving organs, in particular the heart during open-heart surgery, cardiovascular diagnosis or therapeutic intervention. The cardioplegia solutions and compositions comprise a physiological salt solution and optionally one or more of the following compositions: a substrate for the production of ATP, a calcium channel blocker, a vasorelaxant, an antioxidant, an antibiotic, and/or a reagent that buffers intracellular acidity.

In one aspect, the cardioplegia solutions and compositions comprise a physiological salt solution and a substrate for the production of ATP. Optionally, the substrate for the production of ATP is phosphocreatine, creatine ethyl ester, dicreatine malate, creatine gluconate, fructose, sucrose, ribose, hexose or pentose. Alternatively, the substrate for the production of ATP is creatine orotate, creatine monohydrate, adenosine, or dextrose/glucose.

In another aspect, the cardioplegia solutions and compositions comprise a physiological salt solution and a reagent that buffers intracellular acidity. In one aspect, the reagent that buffers intracellular acidity is histidine, glutamine, tryptophan, lysine, or L-taurine. Acidity is also buffered by sodium bicarbonate, Tris-hydroxymethyl aminomethane (THAM), L-carnosine (intracellular acidity), and Beta-alanine. L-carnitine facilitates a decrease in myocardial lactate production, hence reducing acidity. Alternatively, a reagent that buffers intracellular acidity is creatine orotate via facilitated synthesis of carnosine. Creatine monohydrate buffers acidity by increasing energy production and decreasing lactate accumulation.

Optionally, the cardioplegia solutions and compositions comprise a physiological salt and a reagent that quenches reactive oxygen species. In one aspect, the reagent that quenches reactive oxygen species is dithiothreitol (DTT), beta-Mercaptoethanol, Acetylcysteine, Alpha lipoic acid, Taurine, Reserveratrol, Lutein, Selenium, Methionine, or Tocopherols/Vitamin E.

The cardioplegia solutions and compositions prevent ischemic injury. This function is mediated by ascorbic acid, glutathione (reducing agent), carnitine (by preventing accumulation of long chain acyl-CoA that leads to generation of free radicals-ischemic-reperfusion injury), carnosine and alpha lipoic acid, which are free radical (hydroxyl radical, singlet oxygen, peroxyl radical and superoxide) scavengers.

The cardioplegia solutions and compositions contain beta alanine. Beta alanine is an amino acid, which is an agonist next in activity to the cognate ligant glycine, for strychnine-sensitive inhibitory glycine receptors (GlyRs) (the agonist order: glycine>>b-alanine>taurine>>1-alanine, 1-serine>proline). Beta alanine buffers intracellular acidity and pH, improves muscle contraction and increases aerobic threshold.

The intracellular non-bicarbonate buffering capacity of vertebrate muscle is mainly supported by the imidazole groups of histidine residues in proteins, free L-histidine in some fish species, and histidine-containing dipeptides such as carnosine, anserine, and balenine (ophidine) (Abe H, 2000 Biochemistry (Mosc), 65(7):757-65). Results have demonstrated the efficacy of creatine and beta-alanine on strength performance in athletes (Hoffman J et al., 2006 Int J Sport Nutr Exerc Metab, 16(4):430-46).

The cardioplegia solutions and compositions contain L-taurine. L-taurine is a sulfur-containing beta amino acid, which has been implicated in a wide array of physiological phenomena including regulation of heartbeat, osmoregulation, membrane stabilization, preservation of aerobic metabolism, prevention of lactic acidosis, inhibitory neurotransmission, long-term potentiation in the striatum/hippocampus, feedback inhibition of neutrophil/macrophage respiratory bursts, adipose tissue regulation, and calcium homeostasis. Taurine also acts as an antioxidant, and is an endogenous agonist of glycine receptor. An acceptable concentration of taurine in the cardioplegia solutions and compositions is 1-10 mM.

The sulfur-containing amino acid taurine is an inhibitory neuromodulator in the brain of mammals, as well as a key substance in the regulation of cell volumes. The effect of Ca²⁺ on extracellular taurine concentrations is of special interest in the context of the regulatory mechanisms of taurine release. Data imply the involvement of both decreased influx of Ca²⁺ and increased non-specific influx of Na⁺ through voltage-sensitive calcium channels in the regulation of transporter-mediated taurine release in Ca²⁺ depletion (Molchanova S M et al., 2005 Neurochem Int, 47(5):343-9). Moreover, Taurine is observed to act as an antioxidant of peroxynitrite (ONOO⁻) to decrease lipid peroxidation and thus affect liver plasma membrane Na⁺, K+−ATPase by restoring its activity (Kocak-Toker N, et al., 2005 World J Gastroenterol, 11(23):3554-7).

The cardioplegia solutions and compositions optionally contain lacidipine. Lacidipine is a vasorelaxant and calcium channel blocker, which acts on the heart and blood vessels via the nitric oxide/endothelin system. The acceptable range of lacidipine in the cardioplegia solution is 1 pM-1 mM. By contrast, other calcium channel blockers, such as verapamil and nefedipine are effective at 10 μM-1 mM. Lacidipine has a widening effect on blood vessels and slows the movement of calcium through cells, which decreases the rate at which the heart beats, resulting in reduced blood pressure. Lacidipine leads to a significant reduction of the common carotid artery IMT (intima-media thickness) as well as to a decrease in markers of inflammation in patients with coronary artery disease (CAD) during a relatively short period (6 months) (Bae J H et al., 2005 Int J Cardiol, 101(3):377-83). Amlodipine and lacidipine reduce the influence of humoral control and sympathetic autonomic nervous system activity (Zaliunas R et al., 2005 Int J Cardiol, 101(3):347-53). The lipophilic 1,4-dihidropyridine (DHP), lacidipine, is also able to reduce the formation of atheroma plaque in animal models at therapeutic doses. It has potent and long-lasting antihypertensive properties and protects the arterial wall against the development of atherosclerotic lesions in animal models or human subjects with severe and multiple risk factors (Crespi F, 2005 Curr Vasc Pharmacol, 3(2):195-205).

The cardioplegia solutions and compositions optionally contain anandamide, a vasorelaxant. Anandamide (C₂₂H₃₇O₂N), also known as arachidonoylethanolamide, arachidonoylethanolamine, or AEA, is an endogenous cannabinoid neurotransmitter found in animal and human organs, especially in the brain. The acceptable range of anandamide in the cardioplegia solution is 1 nM-1 mM. Results indicate that the coordinated release of sn-2 arachidonylglycerol (2-AG) and anandamide (two endogenous cannabinoids) in the periaqueductal grey matter might mediate opioid-independent stress-induced analgesia (Hohmann A G et al., 2005 Nature, 435(7045):1108-12). Other results are congruent with a hypothesis that anandamide approaches its binding site by laterally diffusing within one membrane leaflet in an extended conformation and interacts with a hydrophobic groove formed by helices 3 and 6 of cannabinoid receptor CB1 while its terminal carbon is closely positioned to a key cysteine residue in helix 6 leading to receptor activation (Tian X et al., 2005 J Biol Chem, 280(33):29788-95). Results using whole-cell patch clamp recordings show that glucocorticoids elicit a rapid, opposing action on synaptic glutamate and gamma-aminobutyric acid (GABA) release onto magnocellular neurons of the hypothalamic supraoptic nucleus and paraventricular nucleus, suppressing glutamate release and facilitating GABA release by activating a putative membrane receptor. Biochemical analysis of hypothalamic slices treated with dexamethasone revealed a glucocorticoid-induced rapid increase in the levels of the endocannabinoids anandamide (AEA) and 2-arachidonoylglycerol (2-AG) (Di S et al., 2005 Endocrinology, 146(10):4292-301). Moreover, recent results suggest that the endocannabinoid 2-AG increases in hepatic ischemia-reperfusion injury of rats, rather than anandamide (Kurabayashi M, et al., 2005 J Invest Surg, 18(1):25-31). It has been proposed that 2-AG exerts its neuroprotection after closed head injury (CHI), at least in part, via CB1 receptor-mediated mechanisms that involve inhibition of intracellular inflammatory signaling pathways (Panikashvili D et al., 2005 J Cereb Blood Flow Metab, 25(4):477-84).

Thus, both lacidipine and anandamide work via cannabinoid receptor CB1 (and CB2; CB1 antagonist SR141716A) by activating eNOS/nNOS on endothelial cells, neurons and other cells. NO interacts with the sympathetic nervous system by inhibition release of norepinephrine from nerves, which leads to vasodialation.

The cardioplegia solutions and compositions optionally contain minocycline. Minocycline is a bacteriostatic antibiotic that is a member of the broad spectrum tetracycline antibiotics. As a result of its long half-life, it generally has serum levels 2-4 times that of most other tetracyclines (150 mg of minocycline results in 16 times the activity level of 250 mg of tetracycline at 24-48 hours). The antibiotic dose is typically up to 200 mg/day. Minocycline in the cardioplegia solution is present at a sub-antibiotic dose. Minocycline inhibits mitochondrial permeability transition (mPT)-mediated cytochrome C release from the mitochondria. The addition of minocycline to the cardioplegia solution during cross clamp protects the heart from acidosis-induced apoptosis.

In mitochondria isolated suspensions, minocycline failed to block superoxide-induced swelling, but was effective in blocking mitochondrial swelling induced by calcium. This latter effect might be mediated through dissipation of mitochondrial transmembrane potential and blockade of mitochondrial calcium uptake (Fernandez-Gomez F J et al., 2005 Neuroscience, 133(4):959-67). Results have demonstrated that systemic administration of the second-generation tetracycline derivative, minocycline, delays the death of axotomized retinal ganglion cells (RGCs) by a mechanism that may be associated with inhibition of microglia activation. The neuroprotective efficacy of minocycline following optic nerve axotomy was superior to that of tetracycline (Baptiste D C et al., 2005 Neuroscience, 134(2):575-82). Evidence suggests that minocycline might exert its anti-inflammatory effect on microglia by inhibiting the expression and release of TNF-alpha, IL-1 beta, and NO (Wang A L et al., 2005 Neurochem Int, 47 (1-2): 152-8).

The cardioplegia solution optionally contains potassium chloride. The potassium concentration in the cardioplegia solution can be varied at the surgeon's discretion over a desired range (0.298 gm/L+/−24-90 mM) without varying the dilution of the cardioplegia solution or the concentration of other ingredients. Alternatively, the potassium and/or other concentrations are varied, while independently varying the degree of dilution and the total flow of cardioplegia solution to the patient's heart. Varying the potassium concentration in the cardioplegia solution allows the perfusionist to minimize the total amount of potassium added to the patient's blood during an operation. In one aspect, a high initial potassium concentration can rapidly arrest the heart and a lower potassium concentration can maintain arrest. The amount of potassium can be adjusted to compensate for the increase in the patient's serum potassium level throughout the course of the operation. Optionally, the potassium concentration of the cardioplegia solution is adjusted in the event of a reoccurrence of heart activity during surgery.

Optionally, the cardioplegia solution contains D-glucose. The presence of D-glucose in the cardioplegia solution is at the discretion of the surgeon. In one aspect, if the patient has diabetes, D-glucose is not included in the cardioplegia solution. Alternatively, if the patient does not have diabetes, D-glucose is included in the cardioplegia solution. D-glucose acts as a vasodilator and a sleep-inducing agent.

The cardioplegia solutions and compositions are used for arresting, protecting and/or preserving organs, in particular the heart during open-heart surgery, cardiovascular diagnosis or therapeutic intervention. In one aspect, the composition contains calcium chloride, potassium phosphate, magnesium sulfate, sodium chloride, sodium bicarbonate, sodium phosphate, adenosine, glutathione, ascorbic acid, L-arginine, L-taurine, L-histidine, L-carnosine, creatine monohydrate, and Beta-alanine. Optionally, the solution also contains one or more of potassium chloride, magnesium chloride, D-glucose, anandamide, minocycline, and lacidipine.

The pH is adjusted to about 6.8 to about 8.0; or about 7.2 to about 7.6. More preferably, the pH is adjusted to about 7.4 using THAM (tromethamine; tris-hydroxymethyl aminomethane), and maintained at 4° C. The osmolarity is maintained at 290-300 mOsM. Preferably, the composition includes the following compounds and concentrations:

| Distilled water | 1.00 | L |
|---|---|---|
| Calcium chloride (1.3 mM) | 0.191 | gm/L |
| Potassium chloride (4.0 mM) | 0.298 gm/L +/−24-90 mM | |
| Potassium phosphate | 0.068 | gm/L |
| (monobasic; 0.5 mM) | | |
| Magnesium chloride | 0.101 gm/L +/−10-20 mM MgCl2 | |
| (hexahydrate; 0.5 mM) | | |
| Magnesium sulfate | 0.123 | gm/L |
| (heptahydrate; 0.5 mM) | | |
| Sodium chloride (130 mM) | 7.597 | gm/L |
| Sodium bicarbonate (5.0 mM) | 0.420 | gm/L |
| Sodium phosphate | 0.050 | gm/L |
| (dibasic; hepatahydrate; 0.19 mM) | | |
| D-Glucose (7 mM) | 1.260 | gm/L* |
| Adenosine (2 mM) | 0.534 | gm/L |
| Glutathione (reduced; 10.0 mM) | 3.073 | gm/L |
| Ascorbic acid (1 mM) | 0.176 | gm/L |
| L-Arginine (5 mM) | 1.073 | gm/L |
| L-Taurine (5 mM) | 0.625 | gm/L |
| Beta-alanine (5 mM) | 0.500 | gm/L |
| L-Histidine (5 mM) | | |
| L-Carnosine (10 mM) | 2.260 | gm/L |
| Creatine monohydrate (2 mM) | 0.298 | gm/L |
| Anandamide (0.01 mM) | 0.003 | gm/L* |
| Minocycline (1.0 mM) | 0.457 | gm/L* |
| Lacidipine | 0.0005 | gm/L* |

The designation “*” denotes that D-Glucose, anandamide, minocycline, and/or lacidipine may be present or absent in the solution based on the discretion of the surgeon and needs of the patient. The perfusionist, surgeon, and/or nurse may manipulate the concentration of potassium and magnesium based on their discretion and the needs of the patient.

A preferred cardioplegia solution includes amounts of the compounds in the following ranges to achieve a desired ratio of compositions:

| Distilled water | 0.01-3.00 | L |
|---|---|---|
| Calcium chloride (1.3 mM) | 0.1-0.5 | gm/L |
| Potassium chloride (4.0 mM) | 0.1-0.5 gm/L +/−24-90 mM | |
| Potassium phosphate | 0.01-0.25 | gm/L |
| (monobasic; 0.5 mM) | | |
| Magnesium chloride | 0.05-0.5 gm/L +/−10-20 mM MgCl2 | |
| (hexahydrate; 0.5 mM) | | |
| Magnesium sulfate | 0.05-0.5 | gm/L |
| (heptahydrate; 0.5 mM) | | |
| Sodium chloride (130 mM) | 5-10 | gm/L |
| Sodium bicarbonate (5.0 mM) | 0.25-0.75 | gm/L |
| Sodium phosphate | 0.01-0.1 | gm/L |
| (dibasic; hepatahydrate; 0.19 mM) | | |
| D-Glucose (7 mM) | 0.5-2.5 | gm/L* |
| Adenosine (2 mM) | 0.25-2.5 | gm/L |
| Glutathione (reduced; 10.0 mM) | 1-5 | gm/L |
| Ascorbic acid (1 mM) | 0.01-0.5 | gm/L |
| L-Arginine (5 mM) | 0.5-2.5 | gm/L |
| L-Taurine (5 mM) | 0.1-1 | gm/L |
| Beta-alanine (5 mM) | 0.1-1 | gm/L |
| L-Histidine (5 mM) | | |
| L-Carnosine (10 mM) | 1-5 | gm/L |
| Creatine monohydrate (2 mM) | 0.1-0.5 | gm/L |
| Anandamide (0.01 mM) | 0.0001-0.005 | gm/L* |
| Minocycline (1.0 mM) | 0.1-1 | gm/L* |
| Lacidipine | 0.00001-0.001 | gm/L* |

The designation “*” denotes that D-Glucose, anandamide, minocycline, and/or lacidipine may be present or absent in the solution based on the discretion of the surgeon and needs of the patient. The pH is adjusted to about 6.8 to about 8.0; or about 7.2 to about 7.6. Preferably, the pH is adjusted to about 7.4 using THAM, and maintained at 4° C. The osmolarity is maintained at 290-300 mOsM.

Anandamide, minocycline, and/or lacidipine may be present or absent in the solution based on the discretion of the surgeon. The perfusionist, surgeon, and/or nurse can manipulate the concentration of potassium and magnesium based on personal preferences.

The compositions for making the cardioplegia solutions are optionally packaged in a kit with the ingredients/amounts listed below or multiples thereof, i.e., scaled up to make 2, 3, 5, 10, 20 times the amount of solution. An exemplary kit contains:

| Distilled water | 0.01-3.00 | L |
|---|---|---|
| Calcium chloride (1.3 mM) | 0.1-0.5 | gm/L |
| Potassium chloride (4.0 mM) | 0.1-0.5 gm/L +/−24-90 mM | |
| Potassium phosphate | 0.01-0.25 | gm/L |
| (monobasic; 0.5 mM) | | |
| Magnesium chloride | 0.05-0.5 gm/L +/−10-20 mM MgCl2 | |
| (hexahydrate; 0.5 mM) | | |
| Magnesium sulfate | 0.05-0.5 | gm/L |
| (heptahydrate; 0.5 mM) | | |
| Sodium chloride (130 mM) | 5-10 | gm/L |
| Sodium bicarbonate (5.0 mM) | 0.25-0.75 | gm/L |
| Sodium phosphate | 0.01-0.1 | gm/L |
| (dibasic; hepatahydrate; 0.19 mM) | | |
| D-Glucose (7 mM) | 0.5-2.5 | gm/L* |
| Adenosine (2 mM) | 0.25-2.5 | gm/L |
| Glutathione (reduced; 10.0 mM) | 1-5 | gm/L |
| Ascorbic acid (1 mM) | 0.01-0.5 | gm/L |
| L-Arginine (5 mM) | 0.5-2.5 | gm/L |
| L-Taurine (5 mM) | 0.1-1 | gm/L |
| Beta-alanine (5 mM) | 0.1-1 | gm/L |
| L-Histidine (5 mM) | | |
| L-Carnosine (10 mM) | 1-5 | gm/L |
| Creatine monohydrate (2 mM) | 0.1-0.5 | gm/L |
| Anandamide (0.01 mM) | 0.0001-0.005 | gm/L* |
| Minocycline (1.0 mM) | 0.1-1 | gm/L* |
| Lacidipine | 0.00001-0.001 | gm/L* |

The designation “*” denotes that D-Glucose, anandamide, minocycline, and/or lacidipine may be present or absent in the solution based on the discretion of the surgeon and needs of the patient. The pH is adjusted to about 6.8 to about 8.0; or about 7.2 to about 7.6. Preferably, the pH is adjusted to about 7.4 using THAM, and maintained at 4° C. The osmolarity is maintained at 290-300 mOsM.

Anandamide, minocycline, and/or lacidipine may be present or absent in the solution based on the discretion of the surgeon. The perfusionist, surgeon, and/or nurse can manipulate the concentration of potassium and magnesium based on personal preferences.

These ingredients packaged together with instructions for use and are mixed in 0.01-2.0 L of distilled water. The kit is packaged or sold without the sterile water component. For example, the kit contains:

| Distilled water | 1.00 | L |
|---|---|---|
| Calcium chloride (1.3 mM) | 0.191 | gm/L |
| Potassium chloride (4.0 mM) | 0.298 gm/L +/−24-90 mM | |
| Potassium phosphate | 0.068 | gm/L |
| (monobasic; 0.5 mM) | | |
| Magnesium chloride | 0.101 gm/L +/−10-20 mM MgCl2 | |
| (hexahydrate; 0.5 mM) | | |
| Magnesium sulfate | 0.123 | gm/L |
| (heptahydrate; 0.5 mM) | | |
| Sodium chloride (130 mM) | 7.597 | gm/L |
| Sodium bicarbonate (5.0 mM) | 0.420 | gm/L |
| Sodium phosphate | 0.050 | gm/L |
| (dibasic; hepatahydrate; 0.19 mM) | | |
| D-Glucose (7 mM) | 1.260 | gm/L* |
| Adenosine (2 mM) | 0.534 | gm/L |
| Glutathione (reduced; 10.0 mM) | 3.073 | gm/L |
| Ascorbic acid (1 mM) | 0.176 | gm/L |
| L-Arginine (5 mM) | 1.073 | gm/L |
| L-Taurine (5 mM) | 0.625 | gm/L |
| Beta-alanine (5 mM) | 0.500 | gm/L |
| L-Histidine (5 mM) | | |
| L-Carnosine (10 mM) | 2.260 | gm/L |
| Creatine monohydrate (2 mM) | 0.298 | gm/L |
| Anandamide (0.01 mM) | 0.003 | gm/L* |
| Minocycline (1.0 mM) | 0.457 | gm/L* |
| Lacidipine | 0.0005 | gm/L* |

The designation “*” denotes that D-Glucose, anandamide, minocycline, and/or lacidipine may be present or absent in the solution based on the discretion of the surgeon and needs of the patient. The pH is adjusted to about 6.8 to about 8.0; or about 7.2 to about 7.6. Preferably, the pH is adjusted to about 7.4 using THAM, and maintained at 4° C. The osmolarity is maintained at 290-300 mOsM.

Anandamide, minocycline, and/or lacidipine may be present or absent in the solution based on the discretion of the surgeon. The perfusionist, surgeon, and/or nurse can manipulate the concentration of potassium and magnesium based on personal preferences.

Optionally, the solution is nano-sized to increase the efficiency of the solution traversing the cellular membrane by any method known in the art, including the method described in U.S. Pat. Nos. 6,521,248 and 7,198,254, which are incorporated herein by reference in their entireties. Nano-sizing refers to the reduction of the particle size to the sub-micron range, with the final particle size typically being 1-10 ηm. The reduction of particle size leads to a significant increase in the efficiency of the solution in traversing the cellular membrane. In one aspect, the efficiency is increased such that at least 20%, at least 25%, at least 50%, at least 75%, or at least 100% of the solution traverses the cellular membrane.

The invention provides for nano-sizing for the solution of the invention prior to use in the methods described herein. Alternatively, the invention provides for nano-sizing the water prior to adding the other compounds/reagents of the solution. In yet another aspect, the invention provides for nano-sizing the water and nano-sizing each compound/reagent of the solution separately prior to mixing in solution.

In one aspect, the composition comprises water packets or water clusters in a nanometer range of size. Optionally, the water packets or water clusters are 1-10 ηm, 1-25 ηm, 25-50 ηm, 50-75 ηm, 75-100 ηm, 100-200 ηm, 200-500 ηm, or 500-999 ηm.

In one aspect, the solutions provided by the invention are administered antegrade. Alternatively, the solutions are administered retrograde.

In another aspect, the solutions presented are mixed with blood prior to use in order to form blood cardioplegia solutions. Optionally, the blood:solution ratio is 5:1, 4:1, 3:1, 2:1 or 1:1. Alternatively, the blood:solution ratio is 1:2, 1:3, 1:4, or 1:5.

What is claimed is:

1) A cardioplegia solution comprising a physiological salt solution further comprising at least one of a composition selected from the group consisting of a calcium channel blocker, a vasorelaxant, a reagent that buffers intracellular acidity, an antioxidant, and an antibiotic.

2) A cardioplegia solution comprising a physiological salt solution and a calcium channel blocker.

3) The cardioplegia solution of claim 2, wherein said calcium channel blocker is lacidipine.

4) A cardioplegia solution comprising a physiological salt solution and a vasorelaxant.

5) The cardioplegia solution of claim 4, wherein said vasorelaxant is anandamide.

6) A cardioplegia solution comprising a physiological salt solution and an agent that buffers intracellular acidity.

7) The cardioplegia solution of claim 6, wherein said agent that buffers intracellular acidity is beta-alanine.

8) A cardioplegia solution comprising a physiological salt solution and an antioxidant.

9) The cardioplegia solution of claim 8, wherein said antioxidant is taurine.

10) A cardioplegia solution comprising a physiological salt solution and an antibiotic.

11) The cardioplegia solution of claim 10, wherein said antibiotic is minocycline.

12) The cardioplegia solution of claim 1, further comprising potassium chloride.

13) The cardioplegia solution of claim 1, further comprising magnesium chloride.

14) The cardioplegia solution of claim 1, further comprising D-glucose.

15) The cardioplegia solution of claim 1, wherein said composition comprises:
0.01-3.00 L distilled water
0.1-0.5 gm/L calcium chloride
0.1-0.5 gm/L potassium chloride
0.01-0.25 gm/L potassium phosphate
0.05-0.5 gm/L magnesium chloride
0.05-0.5 gm/L magnesium sulfate
5-10 gm/L sodium chloride
0.25-0.75 gm/L sodium bicarbonate
0.01-0.1 gm/L sodium phosphate
0.25-2.5 gm/L adenosine
1-5 gm/L glutathione
0.01-0.5 gm/L ascorbic acid
0.5-2.5 gm/L L-Arginine
0.1-1 gm/L L-Taurine
0.1-1 gm/L Beta-alanine
L-Histidine
1-5 gm/L L-Carnosine
0.1-0.5 gm/L creatine monohydrate.

16) The cardioplegia solution of claim 1, wherein said solution does not contain insulin.

17) A kit comprising the cardioplegia solution of claim 1.

18) The cardioplegia solution of claim 1, wherein said solution comprises water clusters in a nanometer range of size.

19) The cardioplegia solution of claim 15, wherein said solution comprises water clusters in a nanometer range of size.

20) The cardioplegia solution of claim 19, wherein said solution comprises water clusters 1-10 ηm in size.

21) The cardioplegia solution of claim 15, wherein said composition further comprises 0.5-2.5 gm/L D-Glucose.

22) The cardioplegia solution of claim 15, wherein said composition further comprises 0.0001-0.005 gm/L anandamide.

23) The cardioplegia solution of claim 15, wherein said composition further comprises 0.1-1 gm/L minocycline.

24) The cardioplegia solution of claim 15, wherein said composition further comprises 0.00001-0.001 gm/L lacidipine.
